(19) United States
(12) Patent Application Publication
(10) Pub. No.: US 2024/0399104 A1
(43) Pub. Date: Dec. 5, 2024

(54) DEVICE AND METHOD FOR PROTECTING URETHRAL DAMAGE DUE TO INADVERTENT INFLATION OF BULB

(71) Applicants: Sunil SINGH, Gurgaon (IN); Anil MANDHANI, Gurgaon (IN)

(72) Inventors: Sunil SINGH, Gurgaon (IN); Anil MANDHANI, Gurgaon (IN)

(21) Appl. No.: 18/688,363

(22) PCT Filed: Sep. 1, 2022
(86) PCT No.: PCT/IN2022/050779

§ 371 Date: Mar. 1, 2024

(30) Foreign Application Priority Data
Sep. 1, 2021 (IN) 202111039659

(51) Int. Cl. A61M 25/00; A61M 25/01; A61M 25/10

(52) U.S. Cl. CPC A61M 25/0017; A61M 25/0102; A61M 25/1018

(57) ABSTRACT

Disclosed is a device (100). The device (100) comprising a catheter shaft (102) such that the catheter shaft (102) has one or more check eyelets (104) adapted to drip urine accumulated in a bladder of a user. The check eyelet (104) drips the urine when, upon insertion of the catheter shaft (102) in the bladder of the user, the check eyelet (104) reaches inside the bladder.

### TECHNICAL FIELD

The present embodiment relates to a catheter to drain urine from the bladder and more particularly to a new design of the catheter that prevents the inadvertent inflation of the bulb in the urethra.

### BACKGROUND

About 15-25% of hospitalized patients may need urethral catheterization (UC) with Indwelling Urinary Catheter during their hospital stay for retention of urine, monitoring of urine output or after any surgical procedure. Significant number of patients in nursing homes and home care facility too, need regular change of the catheter. So, safety of catheterization is an utmost thing to consider.

Iatrogenic Urethral injury (UI) while catheterization is very common. Reported incidence is about 6.7 per 1000 catheterization. In real life practice, the incidence of UI is much more than reported.

Urethral injury caused by a simple procedure of catheterization is preventable but once it is inflicted, it gives a new disease to the patient for his life. Surprisingly, Indwelling Urinary Catheter is in use world over for more than 80 years but there has not been any change in its design to prevent such iatrogenic injuries.

This new design of Indwelling Urinary Catheter called is aimed at reducing the incidence of urethral injury to almost zero.

In urethral catheterization, bulb is inflated using normal saline or distilled water to hold the catheter in bladder. If it is done before the bulb has entered bladder, it can damage the urethra.

Urethral injury is more predominantly reported in males due to the anatomical complexity of male urethra like its length, tortuosity, and presence of enlarged prostate. Published UI literature, accounted for 6% of referral to urology and in a prospective audit incidence of UI was reported to the extent of 13.4 per 1000 male urethral catheterization. Urethral injury in immediate period would result in bleeding requiring transfusion or urosepsis in few patients.

One of the main reasons of UI is an inadvertent inflation of the bulb in the urethra. Even if one notices urine coming out at the other end of the Indwelling Urinary Catheter, bulb may be in the urethra due to inherent design of the catheter. At present Eyelet or a hole in Indwelling Urinary Catheter is located 3 cm distal to the bulb, which may drain urine when the bulb is still in the urethra.

The newly designed catheter, where an additional eyelet is present proximal to the bulb and the distal eyelet is temporarily blocked by an obturator/stylet with a bullous end. Therefore, urine will only be drained with the proximal eyelet, which will ensure that the bulb is inside the bladder eliminating the risk of inflation in the urethra.

Therefore, in light of foregoing discussion, there is a need to overcome the limitations related with the risk involved in pre-inflation of balloon type catheter that causes urethral damage.

### SUMMARY

In one aspect of the present disclosure, a device to protect urethral damage due to inadvertent inflation of a bulb is disclosed. The device includes a catheter shaft having one or more check eyelet adapted to drip urine accumulated in a bladder of a user. The check eyelet drips the urine when the check eyelet reaches inside the bladder upon insertion of the catheter shaft in the bladder of the user.

In some embodiments of the present disclosure, the catheter shaft further includes an outlet adapted to drain the urine from the bladder of the user when the urine drips from the check eyelet.

In some embodiments of the present disclosure, further includes a drainage eyelet and a balloon. The balloon is disposed between the check eyelet and the drainage eyelet. The insertion of the balloon inside the bladder is confirmed when the urine drips from the check eyelet and out of the outlet.

In some embodiments of the present disclosure, further includes a bulb inflation port such that upon insertion of the balloon inside the bladder, the bulb inflation port is adapted to inflate the balloon to hold the device inside the bladder of the user.

In some embodiments of the present disclosure, further includes a stylet that is adapted to block the drainage eyelet to prevent inadvertent inflation of the balloon inside the bladder prior to insertion of the balloon inside the bladder.

In some embodiments of the present disclosure, further includes a thread that passes through the catheter shaft and coupled to the stylet such that the thread, upon being pulled, is configured to unblock the drainage eyelet when the balloon is inserted inside the bladder. When the drainage eyelet is unblocked, at least one of, the check eyelet and the drainage eyelet drips the urine to drain the urine front the bladder.

In another aspect of the present disclosure, a method for draining urine from a bladder of a user is disclosed. The method includes inserting, a device that is provided with a catheter shaft having a check eyelet, inside the bladder of the user. The method further includes confirming, when the check eyelet reaches inside the bladder, a flow of the urine that is accumulated inside the bladder through an outlet of the catheter shaft, to confirm insertion of a balloon of the device inside the bladder. The method further includes inflating, the balloon by way of a bulb inflation port of the device such that upon inflation, the balloon holds the device inside the bladder of the user.

In some embodiments of the present disclosure, the method further includes draining, the urine when the urine drips from the check eyelet, by way of the outlet of the catheter shaft.

In some embodiments of the present disclosure, prior to inserting the balloon inside the bladder, the method further includes blocking, a drainage eyelet of the device by way of a stylet of the device such that blocking of the drainage eyelet prevents inadvertent inflation of the balloon.

In some embodiments of the present disclosure, upon inserting the balloon inside the bladder, the method further includes unblocking the drainage eyelet, by pulling the stylet by way of a thread of the device, wherein the unblocking of the drainage eyelet facilitates dripping of the urine from at least one of, the check eyelet and the drainage eyelet to drain the urine from the bladder.

### BRIEF DESCRIPTION OF DRAWINGS

The drawing/s mentioned herein disclose exemplary embodiments of the claimed invention. Other objects, features, and advantages of the present invention will be apparent from the following description when read with reference to the accompanying drawing.

FIG. 1 illustrates a schematic view of a device, according to an embodiment hemin; and

FIG. 2 illustrates a flowchart that depicts a method or protecting urethral damage by way of the device of FIG. 1, according to an embodiment herein.

To facilitate understanding, like reference numerals have been used, where it would be possible to designate elements common to the figures.

### DETAILED DESCRIPTION

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein. Also, the examples/embodiments described herein should not be construed as limiting the scope of the embodiments herein.

The term “catheter” refers to a flexible tube inserted through a narrow opening into a body cavity, particularly the bladder, for removing fluid.

The term “balloon end” refers to the end of the catheter that includes the balloon. As mentioned, there is a need for the development of a catheter that avoids the urethral damage that may be caused by inflation of balloon in urethra. The embodiment herein overcomes the limitations of the prior art by providing a catheter with two holes on the balloon end, one on the either side of the balloon.

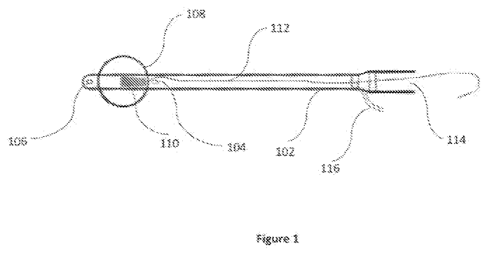

FIG. 1 illustrates a structural view of the device 100, according to an embodiment herein. The device 100 thereinafter interchangeably referred to and designated as “the catheter 100”) includes a catheter shaft 102. The catheter shaft 102 may include one or more check inlets of which a check inlet 104 (hereinafter interchangeably referred to and designated as “the check eyelet 104”) is shown and a drainage inlet 106 (hereinafter interchangeably referred to and designated as “the drainage eyelet 106”). The catheter 100 further includes a balloon 108, an obturator/stylet 110, a thread 112, an outlet 114, and a bulb inflation port 116.

The catheter shaft 102 is adapted to drip urine accumulated in a bladder of a user. Specifically, the catheter shaft 102 drips the urine when the catheter shaft 102 reaches inside the bladder upon insertion of the catheter shaft 102 in the bladder of the user.

The catheter 100 includes the check eyelet 104 and the drainage eyelet 106 on either side of the balloon 108 (hereinafter interchangeably referred to and designated as “the bulb 108”). The bulb 108 is, thus, located between the check eyelet 104 and the drainage eyelet 106. Initially, the bulb 108 is deflated. The obturator/stylet 110 is connected to the thread 112 and blocks the drainage eyelet 106. The thread 112 passes through the catheter shaft 102. In an embodiment, the stylet 110 is a cork stopper. In an embodiment, the stylet 110 is a metal stopper. In an embodiment, the stylet 110 is a rubber stopper. In an embodiment, the stylet 110 is a latex stopper. In an embodiment, the stylet 110 is a plastic stopper.

In an embodiment, the thread 112 is flexible and is a medical grade thin filament which would not break easily. In an embodiment, the thread 112 is a silk or cotton thread. In an embodiment, the thread 112 is a plastic filament.

In an embodiment, the bulb 108 is inflated after insertion of the bulb 108 in the bladder is confirmed, which is confirmed by the draining of urine through the outlet 114, which happens solely from the check eyelet 104, as the drainage eyelet 106 is blocked, thus validating or assuring that the balloon 108 has safely entered the bladder of the subject/user. In an embodiment, the balloon 108 is filled with water. In an embodiment, the balloon 108 is a hollow cylindrical in shape (when it is not inflated). In an embodiment, the balloon 108 is sphere (once it is inflated). In an embodiment, the balloon 108 is oval. In an embodiment, the bulb inflation port 116 is a piston pump. In an embodiment, the bulb inflation port 116 is a one-way valve. After the draining is observed caused by the insertion of the balloon 108 end until at least the check eyelet 104, is in the bladder. The stylet 110 is removed by pulling the thread 112. The balloon 108 is inflated and stylet 110 is removed by pulling the thread 112

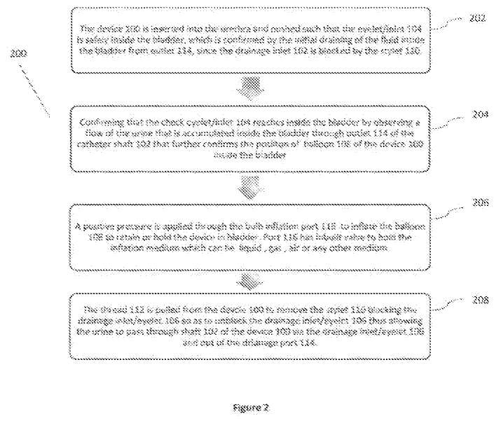

FIG. 2 illustrates a flowchart that depicts working of the catheter of FIG. 1, according to an embodiment herein. The method 200 for draining urine from a urethra of a subject/user by the catheter 100 is provided.

At step 202, the catheter 100 is inserted into the urethra and pushed such that the check eyelet 104 is safely inside the bladder, which is confirmed by the initial draining of the fluid inside the bladder, since the drainage eyelet 106 is blocked by the stylet 110, in an embodiment, the catheter 100 is inserted via the urinal tract. At step 204, confirming that the check eyelet. 104 has reached inside the bladder of the subject/user by observing the flow of urine from the outlet 114.

At subsequent step 206, a positive pressure is applied through the bulb inflation port 116 for inflating the balloon 108 to retain and/or hold the catheter 100 in the bladder.

At step 208, the thread 112 is pulled from the catheter 100 to remove the stylet 110 from the catheter shaft 102 so as to unblock the drainage eyelet 106 thus allowing the urine to pass through the catheter shaft 102 of the catheter 100 via the drainage eyelet 106 and out of the outlet 114.

While the disclosure has been presented with respect to certain specific embodiments, it will be appreciated that many modifications and changes may be made by those skilled in the art without departing from the spirit and scope of the disclosure. It is intended, therefore, by the appended claims to cover all such modifications and changes as fall within the true spirit and scope of the disclosure.

We claim:

1. A device (100) comprising:
a catheter shaft (102) comprising:
one or more check eyelets (104) adapted to drip urine accumulated in a bladder of a user, wherein the one or more check eyelets (104) drip the urine when the one or more check eyelets (104) reach inside the bladder upon insertion of the catheter shaft (102) in the bladder of the user.

2. The device (100) as claimed in claim 1, wherein the catheter shaft (102) further comprising an outlet (114) adapted to drain the urine from the bladder of the user when the urine drips from the one or more check eyelets (104).

3. The device (100) as claimed in claim 1, further comprising:
a drainage eyelet (106); and
a balloon (108) disposed between the one or more check eyelets (104) and the drainage eyelet (106), wherein the insertion of the balloon (108) inside the bladder is confirmed when the urine drips from the one or more check eyelets (104).

4. The device (100) as claimed in claim 3, further comprising a bulb inflation port (116) such that upon insertion of the balloon (108) inside the bladder, the bulb inflation port (116) is adapted to inflate the balloon (108) to hold the device (100) inside the bladder of the user.

5. The device (100) as claimed in claim 3, further comprising a stylet (110) that is adapted to block the drainage eyelet (106) to prevent inadvertent inflation of the balloon (108) inside the bladder prior to insertion of the balloon (108) inside the bladder.

6. The device (100) as claimed in claim 1, further comprising a thread (112) that passes through the catheter shaft (102) and is coupled to the stylet (110) such that the thread (112), upon being pulled, is adapted to unblock the drainage eyelet (106), wherein when the drainage eyelet (106) is unblocked, at least one of the one or more check eyelets (104) and the drainage eyelet (106) drips the urine to drain the urine from the bladder.

7. A method (200) for draining urine from a bladder of a user, the method (200) comprising:
inserting (202), a device (100) that is provided with a catheter shaft (102) having one or more check eyelets (104), inside the bladder of the user;
confirming (204) that the one or more check eyelets (104) reaches inside the bladder by observing a flow of the urine that is accumulated inside the bladder through an outlet (114) of the catheter shaft (102) that further confirms insertion of a balloon (108) of the device (100) inside the bladder; and
inflating (206), the balloon (108) by way of a bulb inflation port (116) of the device (100) such that upon inflation, the balloon (108) holds the device (100) inside the bladder of the user.

8. The method (200) as claimed in claim 7, further comprising draining (208), the urine when the urine drips from the one or more check eyelets (104), by way of the outlet (114) of the device (100).

9. The method (200) as claimed in claim 7, wherein, prior to inserting the balloon (108) inside the bladder, the method (200) further comprising blocking, a drainage eyelet (106) of the device (100) by way of a stylet (110) of the device (100) such that blocking of the drainage eyelet (106) prevents inadvertent inflation of the balloon (108).

10. The method (200) as claimed in claim 7, wherein, upon inserting the balloon (108) inside the bladder, the method (200) further comprising unblocking the drainage eyelet (106), by pulling the stylet (110) by way of a thread (112) of the device (100), wherein the unblocking of the drainage eyelet (106) facilitates dripping of the urine from at least one of the one or more check eyelets (104) and the drainage eyelet (106) to drain the urine from the bladder.

11. A device (100) comprising:
a catheter shaft (102) comprising:
one or more check eyelets (104) adapted to drip urine accumulated in a bladder of a user, wherein the check eyelet (104) drips the urine when the check eyelet (104) reaches inside the bladder upon insertion of the catheter shaft (102) in the bladder of the user;
a drainage eyelet (106);
a stylet (110) that is adapted to block the drainage eyelet (106); and
a thread (112) is coupled to the stylet (110) and passes through the catheter shaft (102) such that the thread (112), upon being pulled, is adapted to unblock the drainage eyelet (106), wherein when the drainage eyelet (106) is unblocked, at least one of the one or more check eyelets (104) and the drainage eyelet (106) drips the urine to drain the urine from the bladder.
